(19) United States
(12) Patent Application Publication
(10) Pub. No.: US 2022/0105356 A1
(43) Pub. Date: Apr. 7, 2022

(54) PORTABLE DEVICE FOR TREATING A PATIENT WITH LIGHT AND METHOD FOR OPERATING THE PORTABLE DEVICE

(71) Applicant: OSRAM Opto Semiconductors GmbH, Regensburg (DE)

(72) Inventor: Daniel Vander LEY, Detroit, MI (US)

(21) Appl. No.: 17/061,212

(22) Filed: Oct. 1, 2020

(51) Int. Cl. A61N 5/06

(52) U.S. Cl. CPC A61N 5/06; A61N 2005/0662; A61N 2005/0659; A61N 2005/0648

(57) ABSTRACT

A portable device configured to treat a patient with light, comprises a light generation unit configured to generate light; a pair of glasses, wherein each of the glasses is configured to be positioned in front of a respective eye of the patient when worn; a light emitting unit situated near or on the glasses and configured to emit light generated by the light generation unit toward a retina of the patient; and a controller unit configured to control the light generation unit in accordance with a programmed script, wherein the programmed script comprises at least one of the following parameters: a wavelength range of the light generated by the light generation unit, an intensity of the light generated by the light generation unit, a duration of a session for treating the patient and a number of sessions for treating the patient.

The invention concerns a portable device for treating a patient with light and a method for operating the portable device.

Light therapy may involve stimulating the retina of a patient by light emission. Light therapy may, for example, help to minimize pain, such as chronic pain.

It is an underlying object of the invention to provide a portable device configured to be worn by a patient proximate to the eyes of the patient and treat the patient with light, wherein the portable device allows to control parameters of the light emission, for example, the wavelength or intensity of the emitted light.

It is further an object of the invention to provide a method for operating the portable device and a system for treating a patient with light including the portable device and a programming device for programming the portable device.

An object underlying the invention is satisfied by a portable device having the features of the independent claim 1. Further, objects underlying the invention are satisfied by a method for operating the portable device having the features of independent claim 15 and a system for treating a patient with light having the features of independent claim 16. Advantageous further developments and aspects of the invention are set forth in the dependent claims.

In a first aspect of the instant application, a portable device configured to be worn by a patient proximate to the eyes of the patient and treat the patient with light is provided. The portable device may have the shape of glasses and may also be denoted as smart glasses.

The portable device comprises a light generation unit, a pair of glasses, a light emitting unit and a controller unit. Further, the portable device may include additional components that are described below in more detail.

Each of the glasses is configured to be positioned in front of a respective eye of the patient when the patient wears the portable device. During operation of the portable device, the light generation unit generates light and the light emitting unit, which is situated near or on the glasses, emits at least a portion of the generated light toward one or both retinas of the patient.

The controller unit controls the light generation by the light generation unit in accordance with a script. The script comprises one or more parameters of the light therapy treatment. The script is programmable and may be programmed by a doctor, a pharmacist, a vendor or any other qualified person. Due to its programmability, the script can be individually tailored to the patient.

The portable device may be configured such that the patient cannot change the programmed script.

The programmed script comprises at least one of the following parameters: a wavelength range of the light generated by the light generation unit, an intensity of the light generated by the light generation unit, a duration of a session for treating the patient and a number of sessions for treating the patient. The programmed script may comprise one or two or three or all four of the aforementioned parameters.

The wavelength range may be a closed interval meaning that the wavelength generated by the light generation unit is restricted to the wavelengths included in the wavelength range. Thus, the light generation unit may only generate light that is within the wavelength range. The wavelength range may have any appropriate size. For example, the wavelength range may be a range from 450 to 490 nm, which means that the light generation unit generates only blue light. Alternatively, the wavelength range may be a range from 490 to 570 nm, which means that the light generation unit generates only green light. In a further example, the wavelength range may be a range from 450 to 570 nm so that the light generation unit generates blue and green light.

The intensity of the light generated by the light generation unit may be tunable and may be, for example, in the range from 0 to 1000 lux. The intensity of the light generated by the light generation unit may be included in the programmed script.

The light therapy treatment may include a number of sessions. During each session light is emitted to one or both retinas of the patient. The light may be emitted constantly during a respective session or in a certain pattern. Each session may have a specific duration. For example, the duration of a session may be given in millisecond increments between 100 ms and 72,000,000 ms. Subsequent sessions may be separated by a pause, in which the light generation unit does not generate light. The duration of each session and the number of sessions may be included in the programmed script.

The glasses may have lenses with or without light attenuating function.

The portable device may comprise a memory unit for storing the programmed script. When the script has been programmed externally, the script can be stored in the memory unit. The controller unit may have access to the memory unit in order to load the programmed script and control the light generation unit in accordance to the light therapy parameters stored in the script.

The portable device may comprise a programming input that can be coupled to an external programming device. The programming device can be used by a doctor or any other qualified person to program the script. Once the script has been programmed, it can be transferred to the portable device via the programming input and can be stored in the memory unit.

The light generation unit may comprise one or a plurality of light sources. If the light generation unit includes several light sources, some of the light sources may emit light having different wavelength ranges.

The light sources may be, for example, light emitting diodes, LEDs, or lasers, in particular laser diodes. The light sources may emit visible light. Each of the light sources may emit light of a certain wavelength or within a certain range of wavelengths, for example, green, blue or red light.

The light emitted by the light sources may be used for stimulating both eyes of the patient. Alternatively, separate light sources may be provided for each eye.

The portable device may comprise a front frame holding the pair of glasses. Further, a pair of side frames, also denoted as earbars, may be provided coupled to opposing ends of the front frame such that, when the portable device is worn by the patient, the side frames extend generally orthogonal to a plane defined by the front frame. The light sources may be integrated into the side frames.

The light emitting unit may comprise at least one light guide that is coupled to the light generation unit and is configured to emit light generated by the light generation unit toward the eyes of the patient. The at least one light guide may be situated near or on the glasses or near or on or in the frame of the glasses.

The at least one light guide may comprise at least one diffuse surface that diffuses the light when exiting the at least one light guide so that diffuse light stimulates the retinas.

The portable device may comprise an on/off switching unit that can be controlled by the patient in order start and/or stop a light therapy session. The on/off switching unit may be an on/off button.

The portable device may comprise a monitoring unit that determines whether the portable device is worn by the patient. The monitoring unit may include a presence sensor and/or an accelerometer and/or a gyroscope to determine placement of the portable device on the patient.

The monitoring unit may deactivate the light generation unit if the monitoring unit determines that the patient does not wear the portable device. Further, the monitoring unit may activate the light generation unit to generate light if the monitoring unit determines that the patient wears the portable device.

When a light therapy treatment is interrupted, for example, because the patient actuates the on/off switching unit to switch off the light generation unit or the monitoring unit detects removal of the portable device from the patient's face, then the state of the light therapy treatment may be stored and the therapy may be continued the next time the portable device is switched on in order to ensure that the treatment, i.e., the full length of the treatment including all sessions, will be completed.

The monitoring unit may be integrated into a bridge that is supported on the nose of the patient and couples the two glasses to each other. The bridge may be a part of the front frame. In this embodiment, the monitoring unit may include a sensor for detecting whether the bridge is proximate to the patient's nose.

The monitoring unit may comprise a light emitter and a light detector. In order to determine whether the patient wears the portable device, the light emitter may emit light, for example infrared light, and the light detector may detect light that has been emitted by the light emitter and has been reflected by the patient's face, for example the nose, to the light detector.

The portable device may comprise a feedback unit that monitors the structure and/or quantity of the light emitted to the patient. For example, the feedback unit may monitor the number of treatment sessions applied to the patient. Further, the feedback unit may monitor the intensity of the light emitted toward the patient. The feedback unit may comprise a light detector, for example, a photodiode, to detect the light emitted by the light emitting unit.

The feedback may be used to make modifications to the generated light to optimize the light toward the prescription parameters. Data about the monitored structure and/or quantity of the light emitted to the patient may be stored in the memory unit and can be loaded by the doctor and another qualified person for further analysis.

The portable device may comprise a wireless transceiver that is configured to transmit and receive data. For example, the transceiver may be based on the Bluetooth standard and/or may exchange data with an application, also denoted as app, running a computer or a mobile device, such as a phone, tablet or watch. The application may be used to activate or initiate a light therapy session, and may further be used to record the time or duration of the light application and/or parametric data for a session. Said data may be uploaded to an online database for tracking.

The portable device may comprise a rechargeable battery that provides power to the light generation unit and the controller unit as well as other electrical components of the portable device, such as the memory unit, the monitoring unit and/or the feedback unit. The battery may be recharged by the patient.

The light therapy applied to the patient by means of the portable device may have various applications. For example, the light therapy may help to minimize pain, such as chronic pain. The light therapy may stimulate endogenous opioid release through the process of controlled light stimulation to the retina.

For managing or minimizing chronic pain, the patient's eyes may be exposed to light with wavelengths in the range from approximately 450 nm to 570 nm. In some embodiments, light of 4 to 1000 lux is administered for a time period of 10 minutes to 8 hours. In some embodiments, the administering is performed for a time period of 3 to 7 days or longer. In some embodiments, the administering is repeated after a gap in time or is continuous.

In a second aspect of the instant application, a method for operating the portable device according to the first aspect is provided. The method comprises that a doctor, a pharmacist, a vendor or any other qualified person programmes the script and the programmed script is transferred to the portable device. Subsequently, the light generation unit is controlled in accordance with the programmed script in order to treat the patient with light generated by the light generation unit.

In a third aspect of the instant application, a system for treating a patient with light is provided. The system comprises the portable device according to the first aspect and a programming device. The doctor or any other qualified person can use software running on the programming device to program the script. The programming device can be coupled to the portable device in order to transfer the programmed script to the portable device.

The following description of figures may further illustrate and explain exemplary embodiments. Components that are functionally identical or have an identical effect are denoted by identical references. Identical or effectively identical components might be described only with respect to the figures where they occur first. Their description is not necessarily repeated in successive figures. There are shown in the drawings:

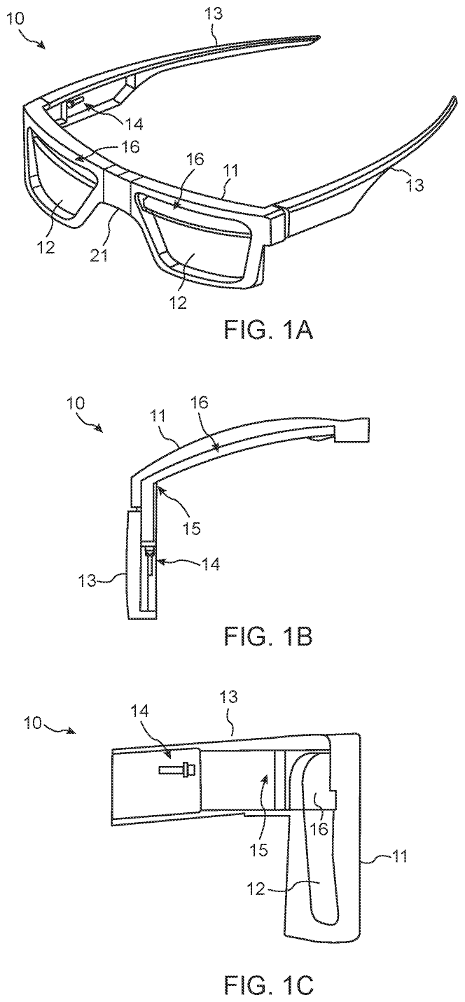

FIG. 1A a three-dimensional representation of a portable device;

FIG. 1B a top view of a section of the portable device;

FIG. 1C a side view of a section of the portable device; and

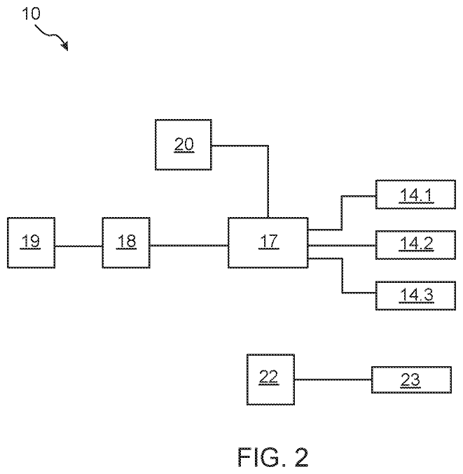

FIG. 2 a schematic circuit diagram of electrical components of the portable device.

FIG. 1A shows a three-dimensional representation of an embodiment of a portable device 10. FIGS. 1B and 1C illustrate top and side views of a section of the portable device 10, respectively.

The portable device 10 includes a front frame 11 holding a pair of glasses 12. A pair of side frames 13 are coupled to opposing ends of the front frame 11 such that, when a patient wears the portable device 10, the side frames 13 extend generally orthogonal to a plane defined by the front frame 11.

A light generation unit in the form of one or more laser diodes or LEDs 14 is integrated into one or both side frames 13.

Light generated by the LEDs 14 is fed into a light-pipe or fiber-optic bundle 15 that is integrated in one or both side frames 13. The plurality of the light guided through the light-pipe or fiber-optic bundle 15 is sent around an approximately 90° turn and mechanically coupled into a larger light guide 16 that is predominantly forward of the patient's eyes and is situated at the upper edge of the glasses 12.

The light guide 16 has a diffuse surface that diffuses the light exiting the light guide 16 towards the patient's eyes and into the retina, where a large percentage of the patient's retina is exposed to the produced light, for example, more than 10% of the retina.

FIG. 2 illustrates a schematic circuit diagram of at least some of the electrical components of the portable device 10. In the embodiment shown in FIG. 2, the light generation unit includes three LEDs 14.1, 14.2 and 14.3 emitting different wavelength ranges. The LED 14.1 emits only red light, the LED 14.2 emits only green light and the LED 14.3 emits only blue light.

A controller unit 17 is coupled to the LEDs 14.1, 14.2, 14.3 and controls them in accordance with a programmed script that is stored in a memory unit 18. The programmed script includes the following parameters of a light therapy treatment: a wavelength range and an intensity of the light used for the light therapy, a duration of a light therapy treatment session and a number of sessions for treating the patient.

The script has been programmed by a doctor, a pharmacist, a vendor or any other qualified person by using an external programming device. The external programming device can be connected to a programming input 19 of the portable device 10. The programming input 19 is coupled to the memory unit 18 and allows to store the programmed script in the memory unit 18.

The portable device 10 further includes a monitoring unit 20 that determines whether the portable device 10 is worn by the patient. For this purpose, the monitoring unit 20 includes a sensor that is integrated in a bridge 21 of the front frame 11 illustrated in FIG. 1. The bridge 21 is supported on the nose of the patient when the patient wears the portable device 10.

When the sensor of the monitoring unit 20 detects that the patient wears the portable device 10, the monitoring unit 20 informs the controller unit 17 accordingly and the controller unit 17 starts the light therapy treatment in accordance with the programmed script that is stored in the memory unit 18. If the patient removes the portable device 10 from the face, the monitoring unit 20 notifies the controller unit 18, which then deactivates the light therapy treatment. The controller unit 17 further saves the state of the light therapy treatment so that the therapy can be continued at the point where it was interrupted, when the portable device 10 is switched on the next time.

In addition, a feedback unit 22 and a photodiode 23 are integrated into the portable device 10. The photodiode 23 is located at a suitable position on the front frame 11 and detects a portion of the light emitted through the diffuse surface of the light guide 11. An output signal of the photodiode 23 is fed to the feedback unit 22. The feedback unit 22 uses the signal received from the photodiode 23 to monitor the structure and/or quantity of the light emitted to the patient. The feedback can be used to make modifications to the generated light to optimize the light toward the prescription parameters.

The description with the aid of the exemplary embodiments does not limit the invention thereto. Rather, the invention comprises any new feature and any combination of features, which in particular includes any combination of features in the patent claims, even if this feature or this combination is not itself explicitly stated in the patent claims or exemplary embodiments.

### LIST OF REFERENCE SIGNS

10 portable device
11 front frame
12 glasses
13 side frame
14 LED
14.1 LED
14.2 LED
14.3 LED
15 light-pipe or fiber-optic bundle
16 light guide
17 controller unit
18 memory unit
19 programming input
20 monitoring unit
21 bridge
22 feedback unit
23 photodiode

1. A portable device configured to be worn by a patient proximate to eyes of the patient and to treat the patient with light, comprising:
a light generation unit configured to generate light;
a pair of glasses, wherein each of the glasses is configured to be positioned in front of a respective eye of the patient when worn;
a light emitting unit situated near or on the glasses and configured to emit light generated by the light generation unit toward retinas of the patient;
a controller unit configured to control the light generation unit in accordance with a programmed script, wherein the programmed script comprises at least one of the following parameters: a wavelength range of the light generated by the light generation unit, an intensity of the light generated by the light generation unit, a duration of a session for treating the patient and a number of sessions for treating the patient; and
a monitoring unit configured to determine whether the portable device is worn by the patient and to activate the light generation unit if the monitoring unit determines that the portable device is worn by the patient and/or to deactivate the light generation unit if the monitoring unit determines that the portable device is not worn by the patient.

2. The portable device as claimed in claim 1, further comprising a memory unit configured to store the programmed script.

3. The portable device as claimed in claim 1, further comprising a programming input configured to be coupled to a programming device in order to receive the programmed script.

4. The portable device as claimed in claim 1, wherein the light generation unit comprises a plurality of light sources and at least some of the plurality of light sources emit light having different wavelength ranges.

5. The portable device as claimed in claim 1, wherein the light emitting unit comprises at least one light guide that is coupled to the light generation unit and is configured to emit light generated by the light generation unit toward the patient.

6. The portable device as claimed in claim 5, wherein the at least one light guide comprises at least one diffuse surface configured to diffuse the light exiting the at least one light guide.

7. The portable device as claimed in claim 1, further comprising an on/off switching unit configured to be controlled by the patient in order start and/or stop a session for treating the patient.

8. (canceled)

9. (canceled)

10. The portable device as claimed in claim 1, further comprising a bridge coupling the two glasses to each other, wherein the bridge is configured to be supported on a nose of the patient when worn and the monitoring unit is integrated into the bridge.

11. The portable device as claimed in claim 1, wherein the monitoring unit comprises a light emitter and a light detector, wherein the light detector is configured to detect light emitted by the light detector in order to determine whether the portable device is worn by the patient.

12. The portable device as claimed in claim 1, further comprising a feedback unit configured to monitor a structure and/or quantity of the light emitted to the patient.

13. The portable device as claimed in claim 1, further comprising a wireless transceiver configured to transmit and receive data.

14. The portable device as claimed in claim 1, further comprising a rechargeable battery configured to provide power to the light generation unit and the controller unit.

15. A method for operating the portable device as claimed in claim 1, comprising:
programming the script;
transferring the programmed script to the portable device; and
control the light generation unit in accordance with the programmed script in order to treat the patient with light generated by the light generation unit.

16. A system for treating a patient with light, comprising:
a portable device as claimed in claim 1; and
a programming device configured to create programmed script and to be coupled to the portable device in order to transfer the programmed script to the portable device.
